(19) United States
(12) Patent Application Publication
(10) Pub. No.: US 2005/0154059 A1
(43) Pub. Date: Jul. 14, 2005

(54) Method of treating type III hypersensitive reaction-related diseases and conditions by using conjugated linoleic acid

(76) Inventors: Mark E. Cook, Madison, WI (US); Dan E. Butz, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/756,719

(22) Filed: Jan. 13, 2004

(51) Int. Cl. A61K 31/202

(52) U.S. Cl. 514/560

(57) ABSTRACT

A method for treating diseases and conditions caused by type III hypersensitive reactions in a human or non-human animal is disclosed. The method involves administering to the animal a conjugated linoleic acid (CLA) or a substance which can be converted to CLA in the animal in an amount effective to reduce inflammation in the animal.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

Conjugated linoleic acid (“CLA”) is a group of positional and geometrical isomers of linoleic acid. Ha Y L et al., Carcinogenesis 8, 1881 (1987); Ha Y L et al., in J. Agric. Food Chem., Vol. 37, No. 1, pp. 75-81 (1987)). These naturally occurring fatty acids are found in beef and dairy products due to ruminal isomerization of linoleic acid. Chin S F et al., Journal of Nutrition 124, 694 (1994). Theoretically, 8 possible geometric isomers of 9,11- and 10,12-octadecadienoic acid (c9,c11; c9,t11; t9,c11; t9,t11; c10,c12; c10,t12; t10,c12 and t10,t12) would form from the isomerization of c9,c12-octadecadienoic acid. As a result of the isomerization, only four isomers (c9, c11; c9,t11; t10,c12; and c10,c12) would be expected. However, of the four isomers, c9,t11- and t10,c12-isomers are predominantly produced during the autoxidation or alkali-isomerization of c9,c12-linoleic acid due to the co-planar characteristics of 5 carbon atoms around a conjugated double-bond and spatial conflict of the resonance radical. The remaining two c,c-isomers are minor contributors.

The relatively higher distribution of the t,t-isomers of 9,11- or 10,12-octadecadienoic acid apparently results from the further stabilization of c9,t11- or t10,c12-geometric isomers, which is thermodynamically preferred, during an extended processing time. Additionally the t,t-isomer of 9,11- or 10,12-octadecadienoic acid that was predominantly formed during the isomerization of linoleic acid geometrical isomers (t9,t12-, c9,t12- and t9,c12-octadecadienoic acid) may influence the final ratio of the isomers or the final CLA content in the samples.

Linoleic acid geometrical isomers also influence the distribution of minor contributors (c,c-isomers of 9,11- and 10,12-, t9,c11- and c11,12-octadecadienoic acids). 5,7; 8,10; and 11,13 isomers might be produced as minor products from c9, c12-octadecadienoic acid or from its isomeric forms during processing.

CLA has been shown to modulate immune response, Cook M E et al., Poult. Sci. 72, 1301 (1993); Chew B P et al., Anticancer Res. 17: 1099 (1997); Miller C C et al., Res. Commun. 198, 1107 (1994), to reduce body fat, Park Y et al., Lipids 32, 853 (1997), and to have anti-carcinogenic and anti-atherosclerotic activities. Ha Y L et al., Carcinogenesis 8, 1881 (1987); Nicolosi R J et al., Artery 22, 266 (1997).

U.S. Pat. No. 6,395,782 disclosed that treating human or non-human animals having autoimmune diseases with CLA can extend the survival time and reduce body weight wasting in these animals. Autoimmune diseases are caused when immune complexes formed between autoantigens and autoantibodies deposit in various tissues and elicit inflammatory responses. Other types of antigen/antibody immune complexes can elicit inflammatory responses similarly. The antigen-antibody immune complex-induced inflammatory responses are called type III hypersensitive reactions as a class. Although CLA has been shown to extend the survival time and to reduce body weight wasting in autoimmune diseases, it is not known whether CLA can relieve any of the symptoms of the autoimmune diseases or diseases caused by type III hypersensitivity in general.

### BRIEF SUMMARY OF THE INVENTION

The present invention relates to a method for treating diseases and conditions caused by type III hypersensitive reactions in a human or non-human animal. The method involves administering to the animal a conjugated linoleic acid (CLA) or a substance that can be converted to CLA in the animal in an amount effective to reduce inflammation caused by the type III hypersensitive reactions in the animal.

### BRIEF DESCRIPTION OF THE SEVERAL VIEWS OF THE DRAWINGS

FIG. 1 shows the effects of CLA on arthritis severity scores in mice with arthritis. “CO Sham” represents the group of sham-injected mice fed with control (corn oil) diet. “CLA Sham” represents the group of sham-injected mice fed with CLA diet. “CO CII” represents the group of mice injected with anti-collagen II antibody and fed with control (corn oil) diet. “CLA CII” represents the group of mice injected with anti-collagen II antibody and fed with CLA diet.

### DETAILED DESCRIPTION OF THE INVENTION

The present invention provides a method for treating a disease or condition caused by a type III hypersensitive reaction where the method includes the step of administering an effective amount of CLA to a human or non-human animal having the disease or condition. An effective amount is defined herein as an amount that can reduce the inflammation caused by the type III hypersensitive reaction in the animal. Type III hypersensitivity occurs as a result of immune complex deposition. Immune complexes are antigen/antibody complexes that form when antigen is produced in excess of antibody. Immune complexes can arise from antigen formed from an infectious agent, an innocuous environmental antigen or an autoantigen cross-reacting with an autoantibody. They can be found at the site of antigen production or in the circulation. Immune complexes are typically cleared by the classical complement pathway or by transfer of immune complexes by red blood cells to the liver or spleen for phagocytosis. The clearing mechanisms can be inadequate when there is excessive production of immune complexes. IgG in immune complexes activates complement as well as macrophages and neutrophils through Fc receptors to cause a hypersensitivity reaction. Complement activation aids in clearing the immune complexes but it also increases the permeability of blood vessels and is chemotactic. Activation of neutrophils, macrophages and platelets cause the release of proteolytic enzymes which damage blood vessels and initiate inflammation. Examples of diseases and conditions as manifestations of type III hypersensitivity include but are not limited to localized Arthus reactions, rheumatoid arthritis, serum sickness, glomerulonephritis, systemic lupus and erythematosus.

Rheumatoid arthritis has been widely used as a model to study type III hypersensitivity due to the availability of good animal models. Using the art-recognized, anti-collagen II antibody-induced mouse arthritis as an example, the inventors have demonstrated that treating the arthritic mice with CLA reduced inflammation induced by type III hypersensitive reactions at one or more joints. The method of the present invention is applicable to all inflammation in animals caused by a type III hypersensitive reaction. The term “animal” or “animals” is used in this application to refer to both human and non-human animals. In particular, the method applies to mammals such as humans, non-human primates, horses, canines, felines, rodents, porcines, bovines, caprines and ovines, especially humans, horses, canines and felines. The invention has particular application in the medical and veterinary fields.

In this application, “conjugated linoleic acid” or “CLA” means an unsaturated fatty acid having 18 carbons and two conjugated double bonds, the fatty acid being selected from the group consisting of 18:2(9c, 11t), 18:2(9t, 11c), 18:2(10c, 12t) and 18:2(10t, 12c), and also including bioactive esters, salts and other chemical derivatives thereof, and mixtures thereof. In addition to CLA, a substance which can be converted to CLA in a human or non-human animal can also be administered in the method of the present invention. An example of such substance is linoleic acid, which can be converted to CLA probably by microorganisms in the gastrointestinal system of an animal (see U.S. Pat. No. 5,827,885 and U.S. Pat. No. 5,837,733, both are incorporated by reference in their entirety). As another example, vaccenic acid (c18:1, 11t), a major fatty acid in milk, can be converted to CLA (18:2 (9c, 11t)) by hepatic delta 9 desaturase after dietary absorption.

The free acid forms of the CLA can be prepared by isomerizing linoleic acid (see, e.g., American Oil Chemists' Society Official Method Cd 7-58, pages 1-11, American Chemists' Society, Champaign, Ill., 1973; U.S. Pat. No. 5,814,663; U.S. Pat. No. 5,208,356, all of which are incorporated by reference in their entirety). The preferred method of synthesizing CLA is alkali isomerization as describe by Chin, et al., Food Composition and Analysis 5: 185-197 (1992). However, CLA may also be isolated from tallow or prepared from linoleic acid by the action of a linoleic acid isomerase from a harmless microorganism such as the Rumen bacterium Butyrivibrio fibrisolvens. Harmless microorganisms such as Lactobacillus reuteri in the intestinal tracts of rats and other monogastric animals may also convert linoleic acid to CLA (S. F. Chin, et al., J. Nutr. 124: 694-701 (1994); U.S. Pat. No. 6,060,304; and U.S. Pat. No. 5,827,885, all of which are incorporated by reference in their entirety).

The CLA obtained by alkali isomerization can contain one or more of the 9,11-octadecadienoic acids and/or 10,12-octadecadienoic acids and active isomers thereof. It may be free or bound chemically through ester linkages. The CLA is heat stable and can be used as is, or dried and powdered. The free acids are readily converted into non-toxic salts, such as the sodium or potassium salts, by reacting chemically equivalent amounts of the free acid with an alkali hydroxide at a pH of about 8 to 9. A specific method for preparing CLA esters is described in U.S. Pat. No. 5,208,356, which is incorporated by reference in its entirety.

The CLA may be administered by any convenient means. For example, the CLA may be formulated for oral, intravenous, intramuscular, transdermal or transmucosal administration. The exact amount to be administered, of course, depends upon the form of CLA employed, the route of administration, species and size of the animal and various other factors. Since CLA is a natural food ingredient and it is relatively non-toxic, the amounts which can be administered in the methods of the invention are not critical as long as they are enough to be effective. Generally, the CLA can be administered in an amount ranging from about 0.001 g/kg to about 1 g/kg of the body weight of a human or non-human animal or higher. This corresponds to about 0.1 g/day to about 40 g/day for a person weighing 45 kg.

Oral delivery is a preferred route for administering CLA in the method of the present invention. For example, CLA can be added to human or non-human animal food. As described above, since CLA is a natural food ingredient and it is relatively non-toxic, the amounts which can be added to the food are not critical as long as they are enough to be effective. In general, the amounts of CLA to be added to a human or non-human animal food can range from about 0.01% to about 5.0% or more, from about 0.05% to about 2.0% or more, or about 0.5% or more by weight of the food.

In one embodiment, the human or non-human animal is fed a food product, such as milk, vegetable oils or egg solids, which have been enriched so that they contain high concentrations of CLA (see, e.g., U.S. Pat. No. 6,113,973 and U.S. Pat. No. 6,060,304, both of which are incorporated by reference in their entirety). In another embodiment, the CLA can be administered to a human or non-human animal in the form of pharmaceutical or veterinary compositions, such as tablets, capsules, solutions or emulsions.

The preferred pharmaceutical and veterinary compositions of CLA contain the non-toxic sodium or potassium salt of CLA in combination with a suitable diluent. When the compositions are solutions or suspensions intended for oral administration the dilutent or ingestible carrier will be one or more dilutents, such as lactose or starch, and the product will be a tablet, capsule or liquid. When the compositions are solutions or suspensions intended for parenteral administration the preferred diluent will be Sterile Water for Injection U.S.P.

An example composition for use in humans is a water in oil fat emulsion, such as Intralipid® (Baxter); Liposyn® (Abbott); Nutrilipid® (McGaw); or SoyaCal® (Alpha Therapeutic), in which about 0.5% to about 2% (preferably 1%) by weight of the oil has been replaced by CLA. These fat emulsions all contain emulsified fat particles of about 0.33-0.5 μm in diameter. In addition about 10% to 20% of the oils which are a mixture of neutral triglycerides of principally unsaturated fatty acids, the emulsions contain Water for Injection USP as a diluent, egg phosphatides (1-2%) as an emulsifying agent and glycerin (2-3%) to adjust toxicity. These emulsions can be infused intravenously to patients requiring parenteral nutrition.

The practice of the present invention is further illustrated by the following non-limiting example.

### EXAMPLE

Materials and Methods

Collagen-induced arthritis (CIA) shares both immunological and pathological features with human rheumatoid arthritis, therefore it has been used extensively as a model to study the pathogenesis of rheumatoid arthritis and for testing therapeutics. Trentham, D. E. et al., J. Exp. Med. 146: 857-868 (1997); Courtenay, J. S. et al., Nature 283: 666-668 (1980); Cathcart, E. S. et al., Lab Invest. 54: 26-31 (1986).

Animals: male BALB/c mice were obtained from Jackson Labs (Bar Harbor, Me.) at four weeks of age. Mice were housed in groups of 3 in a small animal isolation chamber, kept on a twelve hour light dark cycle, and acclimated to their environment for one week. After animals were acclimated to their environment they were randomly assigned to control (0.5% corn oil diet) and treatment groups (0.5% CLA diet). Animals were primed on diet for 3 weeks prior to arthritis induction to ensure tissue saturation of test oils.

Collagen antibody induced arthritis procedure: 2 mg of monoclonal anti-type II collagen antibody cocktail (Chemicon, Temecula, Calif.) in 200 micro-liters PBS was injected i.v. on day one. Forty-eight hours later the inflammatory process was initiated by 50 micrograms of LPS in 200 micro-liters PBS given i.p. Severity of arthritis in the mice was evaluated by a blinded observer scoring each paw from 0 to 4 with a total possible score of 16 per animal (adapted from methods described by Williams, R., M. Feldmann, and R. Maini, Anti-Tumor Necrosis Factor Ameliorates Joint Disease in Murine Collagen-Induced Arthritis. Proc. Natl. Acad. Sci., 89: 9784-9788, 1992, which is herein incorporated by reference in its entirety). Score 0 represents normal paws and limbs. Score 1 represents mild, but definite redness and swelling of the ankle or wrist, or apparent redness and swelling limited to individual digits, regardless of the number of affected digits. Score 2 represents moderate redness and swelling of ankle and wrist. Score 3 represents severe redness and swelling of the entire paw including digits. Score 4 represents maximally inflamed limb with involvement of multiple joints.

Statistical analysis: The SAS statistical package with the Proc Mixed command was used to conduct statistical analysis. Treatment, time and the treatment time interaction were analyzed. Since individuals were measured repeatedly, a test of auto-correlation was included.

Results

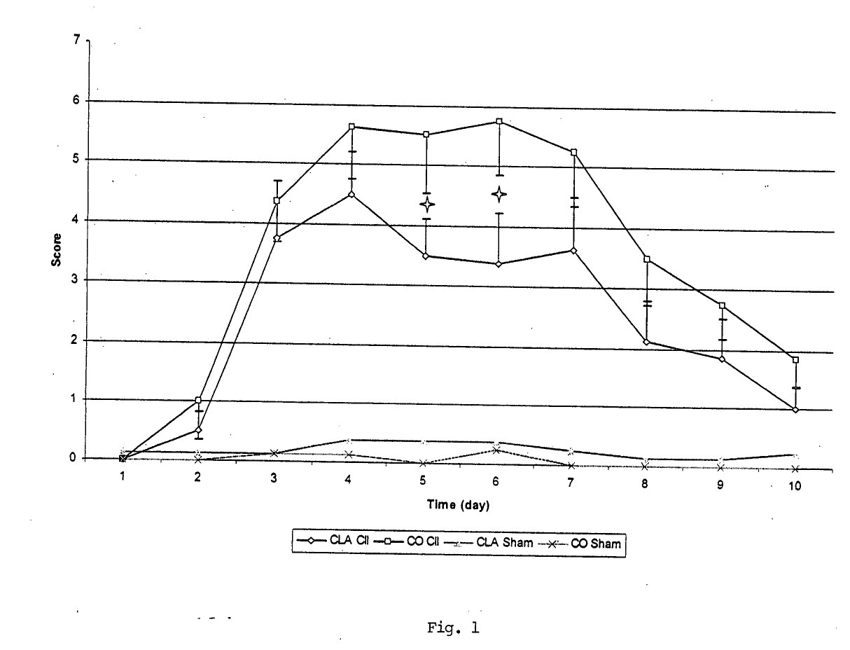

As shown in FIG. 1, arthritis severity scores were significantly higher in the anti-collagen II antibody injected mice as compared to the sham-injected mice. In addition, although elevated, mice fed CLA had significantly lower arthritis severity scores as compared to mice fed corn oil.

The present invention is not intended to be limited to the foregoing example, but to encompass all such modifications and variations as come within the scope of the appended claims.

1. A method of treating diseases or conditions caused by type III hypersensitive reactions in a human or non-human animal, the method comprising the step of administering to the animal a member selected from the group consisting of a conjugated linoleic acid (CLA) and a substance which is converted in the animal to CLA in an amount effective to reduce inflammation caused by the type III hypersensitive reactions in the animal.

2. The method of claim 1, wherein the disease or condition treated is selected from the group consisting of localized Arthus reaction, rheumatoid arthritis, serum sickness, glomerulonephritis, systemic lupus and erythematosus.

3. The method of claim 2, wherein the disease treated is rheumatoid arthritis.

4. The method of claim 1, wherein CLA is administered in the administering step.

5. The method of claim 4, wherein the CLA is selected from the group consisting of a free conjugated linoleic acid, an ester of a conjugated linoleic acid, a non-toxic salt of a conjugated linoleic acid, an active isomer of a conjugated linoleic acid, an active metabolite of a conjugated linoleic acid, and a mixture thereof.

6. The method of claim 5, wherein the free conjugated linoleic acid is selected from the group consisting of an 18:2(9c,11t) isomer, an 18:2(9t,11c) isomer, an 18:2(10c,12t) isomer and an 18:2 (10t,12c) isomer.

7. The method of claim 1, wherein the animal is selected from the group consisting of a mammal and an avian.

8. The method of claim 7, wherein the mammal is selected from the group consisting of a human, a non-human primate, a horse, a canine, a feline, a rodent, a porcine, a bovine, a caprine and an ovine.

9. The method of claim 8, wherein the mammal is selected from the group consisting of a human, a horse, a canine and a feline.

10. The method of claim 9, wherein the mammal is a human.

11. The method of claim 1, wherein the administering step comprises a method selected from the group consisting of oral delivery, intramuscular injection, intravenous injection, transdermal delivery, transmucosal delivery and parenteral delivery.

12. The method of claim 11, wherein the administering step comprises oral delivery.

13. The method of claim 12, wherein the CLA is added to a food and the food is consumed by the animal.

14. The method of claim 13, wherein the food contains 0.01% to 5% of CLA by weight of the food.

15. The method of claim 14, wherein the food contains 0.05% to 2% of CLA by weight of the food.

16. The method of claim 1, wherein the CLA is administered in a dosage of between about 0.001 g/kg and 1 g/kg body weight of the animal.
